(19) United States
(12) Patent Application Publication
(10) Pub. No.: US 2009/0112172 A1
(43) Pub. Date: Apr. 30, 2009

(54) Audio alert diapers

(76) Inventor: Linda Lee Nicewander Tooker, Delrio, TX (US)

Correspondence Address: Linda Lee Nicewander Tooker, 1404 Qualia Drive (Rear), Delrio, TX, 78840, US

(21) Appl. No.: 11/789,430

(22) Filed: Oct. 10, 2007

Related U.S. Application Data
(60) Provisional application No. 60/794,160, filed on Apr. 24, 2006.

(51) Int. Cl. A61F 13/42

(52) U.S. Cl. 604/361

(57) ABSTRACT

The subject of this patent pending idea, the AUDIO ALERT DIAPERS, consist of SEVERAL INNER LAYERS OF VARYING MATERIAL TYPES. Some for absorbancy and the remaining layers present serve as conduction contact area(S), so when moisture is present, these areas act as poles of a switch to activate a musical circuit. The conducting layers are separated by absorbant material, so that only moisture activates the audio. The purpose is to signal a listener when ample/excessive moisture is present. In summary, the diaper contains moisture until and excessive amount is present and alerts a listener. At the end of the said disposable diaper's absorbancy, a deactivation pull tab will disable the audio produced by the circuit.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR COMPUTER PROGRAM LISTING COMPACT DISC APPENDIX

Not Applicable.

### BACKGROUND OF THE INVENTION

Commonly, diaper rash or irritation occurs due to the extended time between an accident, or urination, and the cleaning process. Factors such as the mother/healthcare assistant having no knowledge of the incident, or patient's state of being, contribute to the delay in time. Moreover, the distress experienced by the child/patient is directly related to the “time factor”. The time factor is the amount of time between the urination and cleanup.

Evidence (not cited here due to extended and varied research) proves music enhances all or most experiences for people in some way. Generally speaking, music played for one of the above mentioned children and or patients could decrease the amount of perceived distress by measure of the soothing potency of the music.

Also, sleep habits of the child and/or patient have been negatively affected by the occurrence of urination. And given that the appropriate action is taken by the responsible party when notified of such an occurrence sanitary issues can be lessened and music can be a positive reinforcement for the child/patient.

### BRIEF SUMMARY OF THE INVENTION

The Audio Alert Diaper troubleshoots several existing problems in healthcare. In general the diaper itself works as a switch to trigger an audio response of varying types to inform and sooth those concerned when moisture is present.

These diapers upon reaching the end of their useful absorbency will sound off in melody. This can target three areas to improve domestic healthcare problems: 1) Gain the attention of the responsible party in which case the appropriate actions are taken to lower the time factor, 2) Significantly reduce the amount of distress perceived by the child/patient, 3) Improve sleep quality and/or time of both the responsible party and the child/patient.

### BRIEF DESCRIPTION OF THE SEVERAL VIEWS OF THE DRAWINGS

The figures, with the exception of FIG. 2, represent interior views of materials and/or modules organized with the invention.

Figure one illustrates by layer the suggested material types and their respective organization which compose the invention.

FIG. 2 is the electronic schematic representing the circuit.

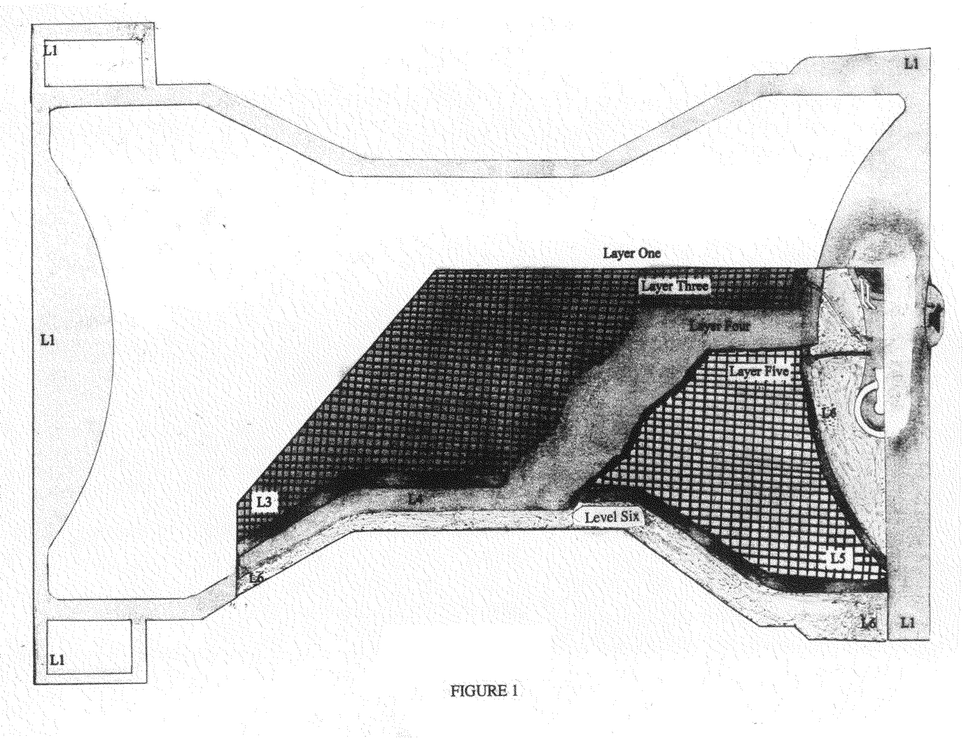

FIGS. 3 and 4 represent the exterior of the circuit module with FIG. 3 displaying a top view and FIG. 4 displaying a side view. Both of these FIGURES contain views of the component labeled “S2.”

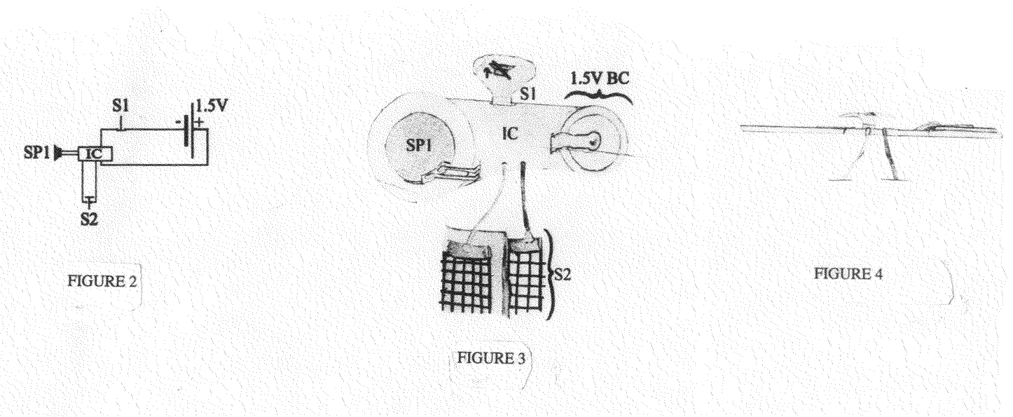

### DETAILED DESCRIPTION OF THE INVENTION

FIG. 1 illustrates by layer the suggested material types and their respective organization which make up the invention. The layers are defined, and subject to slight variation as follows:
1. Layer one is a smoothly textured paper product used to line the diapers inner fold exterior, which is the material which will come in contact with the skin.
2. Layer two (not graphically illustrated) is a layer of paper and/or cotton used to absorb a percentage of the total moisture.
3. Layer three is the first pole of switch number two (S2). The material is a metal conductor in the form of a fine screen.
4. Layer four is another layer composed of paper and/or cotton which will absorb the majority of the moisture contained withing the diaper.
5. Layer five is the second of two pole for switch number two (S2), also a finely threaded, metallic, conducting screen.
6. Layer six is the outer fold exterior which will face outwards and is sealed at the edges with layer one being larger but similar in shape to layers 2 through 5 (inner layers). Layer six along with layer one make up the total visible exterior of the diaper.
   External Features: The backside hem of the garment features a deactivation pull tab. This will disable the audio produced by the circuit.

This diaper serves as a switch which closes a circuit containing a melody producing silicone chip/IC when ample moisture is present. The construct of the circuit is identified by FIGURES three, four and five. This circuit is composed of four basic components and one custom manufactured component:
S1 This is a Normally Closed, Single Pole-Single Throw switch, or NC-SPST. This switch serves to break the connection therefor deactivating the circuit when it is in “alert mode” (playing sound).
S2: This is a Normally Open, Single Pole-Single Throw switch, or NO-SPST. This switch is comprised of two conducting screens, separated by a layer of paper and/or cotton which functions as a closed switch only when moisture is present.
1.5 v Button Cell (BC): This cell commonly found in small lighting devices, watches, electronic thermometers, etc. is lightweight, very flat and provides a surplus of power for the low current load.
SP1: This speaker available in many retail forms produces beeps, chirps and other orchestral basic harmonics. Thinner than the button cell and even lighter in weight.
Custom IC: This integrated circuit (not defined here due to complexity) generates the melody (melodies) which is triggered by S2 and deactivated by S1. This silicone chip will repeat the melody with a time gap in-between each play.

1. I claim a disposable diaper with two layers of fine metallic conducting screen between the other layers in the diaper, having a wire connected to each of the two fine metallic conducting screens, which make the switch activate the music chip/circuit when ample moisture is present and at the end of the said disposable diaper's absorbancy, a deactivation pull tab will disable the audio produced by the circuit.
